(19) United States
(12) Patent Application Publication
(10) Pub. No.: US 2017/0224849 A1
(43) Pub. Date: Aug. 10, 2017

(54) MICROCAPSULES AND USES THEREOF

(71) Applicant: President and Fellows of Harvard College, Cambridge, MA (US)

(72) Inventors: Nichlaus James Carroll, Belmont, MA (US); Maximilian Zieringer, Cambridge, MA (US); David A. Weitz, Bolton, MA (US); Joseph D. Brain, Essex, MA (US); Nagarjun Konduru Vendata, Quincy, MA (US); Ramon Molina, Weymouth, MA (US); Rajiv Gupta, Wayland, MA (US)

(21) Appl. No.: 15/519,288

(22) PCT Filed: Oct. 13, 2015
(86) PCT No.: PCT/US15/55315

§ 371 Date: Apr. 14, 2017

Related U.S. Application Data
(60) Provisional application No. 62/063,556, filed on Oct. 14, 2014.

(51) Int. Cl. A61K 49/00; A23L 33/10; A23P 10/30; A61K 49/04; B01J 13/14

(52) U.S. Cl. CPC A61K 49/0091; A61K 49/0043; A61K 49/0054; A61K 49/0028; A61K 49/0438; A61K 49/048; B01J 13/14; A23P 10/30; A23L 33/10; A23V 2002/00

(57) ABSTRACT

Certain aspects of the present invention relates to microcapsules comprising a core; and a hydrophobic, cross-linked polymeric shell, as well as method for making and using same. Some embodiments of the present invention relate to microcapsules comprising a core; and a hydrophobic, cross-linked polymeric shell. These microcapsules can be used in a variety of applications, including agriculture, encapsulation of food ingredients, health care, cosmetics (e.g., perfumes, detergents, and sunscreen), coatings (e.g., paints and pigments), additives, catalysis, and oil recovery.

### RELATED APPLICATIONS

This application claims the benefit of U.S. Provisional Patent Application Ser. No. 62/063,556, filed Oct. 14, 2014, entitled “Microcapsules and Uses Thereof,” incorporated herein by reference in its entirety.

### GOVERNMENT FUNDING

This invention was made with government support under Grant Nos. W81XWH-13-2-0067, W81XWH-10-1-1043, W81XWH-09-02-0001 and N66001-11-1-4204 awarded by the Department of Defense, and Grant No. R01 DK052625-14 awarded by the National Institutes of Health. The government has certain rights in the invention.

### BACKGROUND

Microcapsules can comprise a core and polymeric shell. Typically, the core is either liquid or solid and may contain, in some cases, actives. The shell may be made up of a polymeric network. The shell acts as a barrier to keep actives separated from the microcapsules' exterior. Although microcapsules hold great potential for applications involving the encapsulation and triggered release of actives for application in agriculture, encapsulation of food ingredients, health care, cosmetics, coatings (e.g., paints and pigments), additives, catalysis, and oil recovery, the leakage of actives from microcapsules is typically observed and presents a technological challenge for their practical application.

### SUMMARY

Certain embodiments of the present invention are directed to fabricated cross-linked polymeric shells that substantially prevent encapsulated actives from leaking. This may solve the problem of microcapsule leakage in accordance with some embodiments. In some embodiments, the release of the actives from the cross-linked polymeric shells can be triggered by an external trigger. The various advantages of some of the microcapsules described herein, which are made using some of the methods described herein, include one or more of: chemical inertness; long-term stability independent of external pH; high mechanical stability; high encapsulation efficiency; high cargo diversity (hydrophobic or hydrophilic actives); large core-shell ratio (which may result in thin shells, which, in turn, can allow high loading of actives per microcapsule, thus greatly reducing the amount of shell material); highly efficient long-term storage of encapsulated actives in the core; can be made and stored in organic or aqueous media; and/or highly defined and highly controllable release mechanisms, which may result in the reduction of unwanted release of the microcapsule “payload” prior to triggering release, if release is desired.

The subject matter of the present invention involves, in some cases, interrelated products, alternative solutions to a particular problem, and/or a plurality of different uses of one or more systems and/or articles.

In one aspect, the present invention is generally directed to a microcapsule comprising a core and a hydrophobic, cross-linked polymeric shell.

In another aspect, the present invention is generally directed to a microcapsule comprising a core comprising an emulsion, and a polymer shell surrounding the core.

The present invention, in yet another aspect, is generally directed to a microcapsule comprising a core, and a polymer shell surrounding the core, where the polymer shell comprises particles.

In still another aspect, the present invention is generally directed to a microcapsule comprising a core, and a polymer shell surrounding the core, where the polymer shell comprises cross-linked perfluoropolyether.

The present invention, in another aspect, is generally directed to a method of forming a microcapsule. In some embodiments, the method comprises providing or obtaining a double emulsion comprising a first aqueous phase comprising a surfactant; an organic phase comprising a hydrophobic, cross-linkable polymer, and a second aqueous phase optionally comprising an active, and cross-linking the hydrophobic, cross-linkable polymer to form a hydrophobic, cross-linked polymeric shell substantially surrounding a core.

In another aspect, the method includes producing a double emulsion comprising an inner phase comprising a preformed emulsion, a middle phase comprising a polymer and containing the inner phase, and an outer phase containing the middle phase, and polymerizing the polymer of the middle phase to produce a microcapsule containing the preformed emulsion.

In another aspect, the present invention encompasses methods of making one or more of the embodiments described herein. In still another aspect, the present invention encompasses methods of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is an electron micrograph of the microcapsules of some of the embodiments of the present invention;

FIG. 2 is a scheme showing one example of a method of making the microcapsules of certain embodiments of the present invention;

FIG. 3 is an example scheme showing the synthesis of a perfluoropolyether dimethylacrylate compound (panel (a)) and contact angles (a measure of the surface energy/hydrophobicity) observed for such compounds (panel (b));

FIG. 4 shows photographs (panels (a) and (b)) of microcapsules of certain embodiments of the present invention filled with Allura Red dye and plots of leakage data (panels (c) and (d));

FIG. 5 is a table summarizing leakage data for various encapsulating materials, including the material used to form the hydrophobic, cross-linked polymeric shell of the microcapsules of some embodiments of the present invention, e.g., PFPE acrylate; and

FIG. 6 is a plot of percent “cargo” released as a function of time for microcapsules of some embodiments of the present invention when such microcapsules are exposed to osmotic stress.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

### DETAILED DESCRIPTION

Certain aspects of the present invention relates to microcapsules comprising a core; and a hydrophobic, cross-linked polymeric shell, as well as method for making and using same.

Some embodiments of the present invention relate to microcapsules comprising a core; and a hydrophobic, cross-linked polymeric shell. These microcapsules can be used in a variety of applications, including agriculture, encapsulation of food ingredients, health care, cosmetics (e.g., perfumes, detergents, and sunscreen), coatings (e.g., paints and pigments), additives, catalysis, and oil recovery.

The microcapsules may have any suitable dimensions and are, in some embodiments, substantially spherical. But the microcapsules may also be of any suitable shape, including oblong and/or other non-spherical shapes. In some embodiments, the microcapsules may be substantially spherical and may have a diameter of from about 0.1 micrometers to about 1000 micrometers, e.g., from about 0.1 micrometers to about 500 micrometers, from about 5 micrometers to about 500 micrometers, from about 5 micrometers to about 250 micrometers, from about 50 micrometers to about 300 micrometers, from about 100 micrometers to about 300 micrometers, from about 50 micrometers to about 150 micrometers, from about 50 micrometers to about 100 micrometers, from about 500 micrometers to about 1000 micrometers, from about 350 micrometers to about 800 micrometers or from about 250 micrometers to about 750 micrometers.

In some cases, the microcapsules may have an average cross-sectional diameter of less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers, or between about 50 micrometers and about 1 mm, between about 10 micrometers and about 500 micrometers, or between about 50 micrometers and about 100 micrometers in some cases. The average cross-sectional diameter may also be at least about 1 micrometer, at least about 2 micrometers, at least about 3 micrometers, at least about 5 micrometers, at least about 10 micrometers, at least about 15 micrometers, or at least about 20 micrometers in certain cases. In some embodiments, at least about 50%, at least about 75%, at least about 90%, at least about 95%, or at least about 99% of the microcapsules within a plurality of microcapsules has an average cross-sectional diameter within any of the ranges outlined in this paragraph.

The hydrophobic, cross-linked polymeric shell has any suitable thickness. In some embodiments, the shell has a thickness of from about 20 nm to about 10 micrometers, about 200 nm to about 10 micrometers, about 200 nm to about 750 nm, from about 200 nm to about 1 micrometers, from about 750 nm to about 5 micrometers, from about 1 micrometers to about 5 micrometers or from about 2 micrometers to about 5 micrometers.

In certain aspects, the shell may have an average thickness of less than about 1 micrometer, less than about 500 nm, less than about 300 nm, less than about 200 nm, less than about 100 nm, less than about 50 nm, less than about 30 nm, less than about 20 nm, or less than about 10 nm. The thickness may be determined, for example, optically or visually, or in some cases, may be estimated based on the volumes and/or flowrates of fluid entering or leaving a conduit. If the microcapsule is non-spherical, then average thicknesses or diameters may be determined or estimated in some cases using a perfect sphere having the same volume as the non-spherical microcapsule or microcapsule interiors.

The core of the microcapsules of some embodiments have any suitable volume. In some embodiments, the volume is such that the microcapsules have a v/v core-shell ratio of about 1:2 to about 1:0.1, e.g., from about 1:1 to about 1:0.1, from about 1:0.9 to about 1:0.1 or from about 1:0.8 to about 1:0.5.

It should also be understood that in some cases, the core contained within the shell is relatively large, e.g., a large percentage of the volume of the microcapsule is taken up by the core, which may result in the shell having a relatively thin thickness, as discussed above. Thus, for example, on a volume basis, the core may take up at least about 80% of the volume of the microcapsule, and in some cases, at least about 85%, at least about 90%, at least about 95%, at least about 97%, at least about 98%, at least about 99%, at least about 99.5%, or at least about 99.7% of the volume of the microcapsule. In some cases, the diameter of the core may be at least about 80% of the diameter of the microcapsule, and in some cases, at least about 85%, at least about 90%, at least about 95%, at least about 97%, at least about 98%, at least about 99%, at least about 99.5%, or at least about 99.7% of the diameter of the microcapsule.

In some embodiments, the microcapsules exhibit a percent leakage of less than 2% over a period of about 30 days, e.g., less than 1.5%, less than 1%, less than 0.5% or less than 0.1% over a period of about 30 days. In some embodiments, the encapsulation efficiency observed for the microcapsules is 60% or greater, greater than 70%, greater than 80%, greater than 90%, greater than 95%, greater than 98% or greater than 99%. In some embodiments, the encapsulation efficiency of the microcapsules is from about 60% to about 100%, from about 70% to about 95%, from about 75% to about 95%, from about 80% to about 95%, from about 90% to about 100%, from about 95% to about 99% or from about 95% to about 98%.

In some aspects of the invention, at least a portion of a double or other multiple emulsion droplet may be solidified to form a particle or a capsule, for example, containing an inner fluid and/or a species as discussed herein. A fluid, e.g., within an outermost layer of a multiple emulsion droplet, can be solidified using any suitable method. For example, in some embodiments, the fluid may be dried, gelled, and/or polymerized, and/or otherwise solidified, e.g., to form a solid, or at least a semi-solid. The solid that is formed may be rigid in some embodiments, although in other cases, the solid may be elastic, rubbery, deformable, etc. In some cases, for example, an outermost layer of fluid may be solidified to form a solid shell at least partially containing an interior containing a fluid and/or a species. Any technique able to solidify at least a portion of a fluidic droplet can be used. For example, in some embodiments, a fluid within a fluidic droplet may be removed to leave behind a material (e.g., a polymer) capable of forming a solid shell. In other embodiments, a fluidic droplet may be cooled to a temperature below the melting point or glass transition temperature of a fluid within the fluidic droplet, a chemical reaction may be induced that causes at least a portion of the fluidic droplet to solidify (for example, a polymerization reaction, a reaction between two fluids that produces a solid product, etc.), or the like. Other examples include pH-responsive or molecular-recognizable polymers, e.g., materials that gel upon exposure to a certain pH, or to a certain species. In some embodiments, a fluidic droplet is solidified by increasing the temperature of the fluidic droplet. For instance, a rise in temperature may drive out a material from the fluidic droplet (e.g., within the outermost layer of a multiple emulsion droplet) and leave behind another material that forms a solid. Thus, in some cases, an outermost layer of a multiple emulsion droplet may be solidified to form a solid shell that encapsulates one or more fluids and/or species.

For example, the hydrophobic, cross-linked polymeric shell can comprise any suitable hydrophobic, cross-linkable (e.g., polymerizable) polymer that can be subsequently cross-linked (e.g., polymerized) via any suitable means for cross-linking, thereby yielding a hydrophobic, cross-linked (e.g., polymerized) polymeric shell. Examples of suitable hydrophobic, cross-linkable polymers include, but are not limited to, polymers comprising cross-linkable perfluoropolyether (PFPE) blocks that are end-capped with a suitable cross-linking group (e.g., end-capped with methacrylate groups; see, e.g., Scheme I, below). Without being bound by any particular theory, it is believed that the PFPE block confers chemical inertness and hydrophobicity to the microcapsule shell. In addition, cross-linkable groups, such as photo-curable acrylate groups, facilitate a highly cross-linked homogeneous polymeric network.

It has been surprisingly found that at least the combination of polymers comprising cross-linkable perfluoropolyether (PFPE) blocks and photocurable acrylate groups minimizes (e.g., eliminates) the formation of pores in the hydrophobic, cross-linked polymeric shell, while reducing the effect of polymer swelling because of the high degree of hydrophobicity afforded by the PFPE blocks. But, even though the number of pores is reduced, the microcapsules of some embodiments have shown excellent gas permeability so that, for example, if the core of the microcapsule comprises an evaporable solvent (e.g., water, methanol, ethanol, isopropanol, ethyl acetate, dichloromethane, chloroform, benzene, toluene, hexane, and tetrahydrofuran (THF)), the microcapsules can be exposed to conditions under which the solvent can be evaporated through the shell, without compromising the integrity of the shell (e.g., the shell still does not leak a substantial amount of any material that remains in the core). Conditions under which the solvent can be evaporated through the shell include, but are not limited to, at least one of reduced pressure, vacuum, ambient conditions, freeze drying, and elevated temperatures.

In some embodiments, suitable hydrophobic, cross-linkable polymers include, but are not limited to polymers comprising one or more repeating polyfluoro ethylene oxide units (i.e., —CFₙH₂₋ₙFₘH₂₋ₘO— units, wherein each n and m, at each occurrence are each, independently 1 or 2) and/or one or more repeating fluoromethyleneoxide units (i.e., —CF_{q}H_{2-q}O— units, wherein each q, at each occurrent, is 0, 1 or 2). In some embodiments, the resulting polymer shell is a fluorinated polymeric shell. In some embodiments, the fluorinated polymeric shell comprises up to about 60 mol % fluorine, e.g., about 1 mol % to about 60 mol % fluorine, about 5 mol % to about 50 mol % fluorine, about 10 mol % to about 50 mol % fluorine, about 5 mol % to about 25 mol % fluorine, about 10 mol % to about 40 mol % fluorine or about 25 mol % to about 50 mol % fluorine.

In some embodiments, the fluorinated polymeric shell comprises from about 30 to about 60 mol % tetrafluoroethylene units, e.g., from about 35 to about 55 mol %, from about 40 to about 50 mol % or from about 45 to about 55 mol % tetrafluoroethylene units. In some embodiments, the fluorinated polymeric shell comprises about 49 mol % tetrafluoroethylene units. In some embodiments, the fluorinated polymeric shell comprises from about 30 to about 60 mol % difluoromethylene units, e.g., from about 35 to about 55 mol %, from about 40 to about 50 mol % or from about 45 to about 55 mol % difluoromethylene units. In some embodiments, the fluorinated polymeric shell comprises about 49 mol % difluoromethylene units.

In some embodiments, the fluorinated polymeric shell comprises from about 30 to about 60 mol % tetrafluoroethylene units, e.g., from about 35 to about 55 mol %, from about 40 to about 50 mol % or from about 45 to about 55 mol % tetrafluoroethylene units; and from about 30 to about 60 mol % difluoromethylene units, e.g., from about 35 to about 55 mol %, from about 40 to about 50 mol % or from about 45 to about 55 mol % difluoromethylene units. In some embodiments, the fluorinated polymeric shell comprises about 49 mol % tetrafluoroethylene units and about 49 mol % difluoromethylene units.

The hydrophobic, cross-linkable polymer comprises cross-linkable groups that can be subsequently cross-linked via any suitable means for cross-linking, in certain embodiments. The cross-linkable groups may be cross-linked by, e.g., radical polymerization, anionic polymerization, cationic polymerization, ring-opening polymerization, polycondensation, click reactions or Michael additions.

In some embodiments, the hydrophobic, cross-linkable polymer comprises a compound of the formula (I):

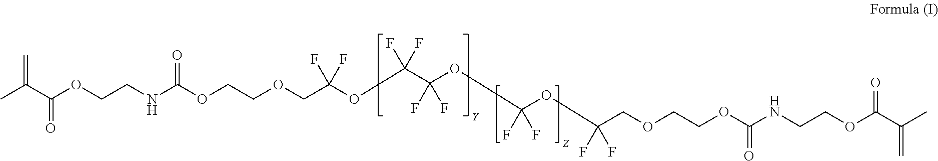

wherein Y and Z are each, independently, about 5 to about 50, e.g., from about 5 to about 25, from about 10 to about 50, from about 10 to about 25, from about 15 to about 30, from about 15 to about 25 or from about 10 to about 20. In some embodiments Y and Z are each, independently, about 20. Compounds of the formula (I) comprise repeating tetrafluoro ethylene oxide units, repeating difluoromethyleneoxide units, and acrylate cross-linking groups. In one example, compounds of the formula (I) can be cross-linked (i.e., polymerized) via radical chemistry in the presence of a radical initiator (e.g., ammonium peroxodisulfate, dibenzoyl peroxide, 2,2-dimethoxy-2-phenylacetophenone, and mixtures thereof).

An example of a method for synthesizing the compounds of the formula (I) is shown in Scheme I, below, wherein Novec™ 7100 (methoxy nona-fluorobutane, “engineered fluid” from 3M) and THF comprises a non-limiting solvent system that may be utilized to synthesize the compounds of the formula (I); and the variables X and Y are as defined herein:

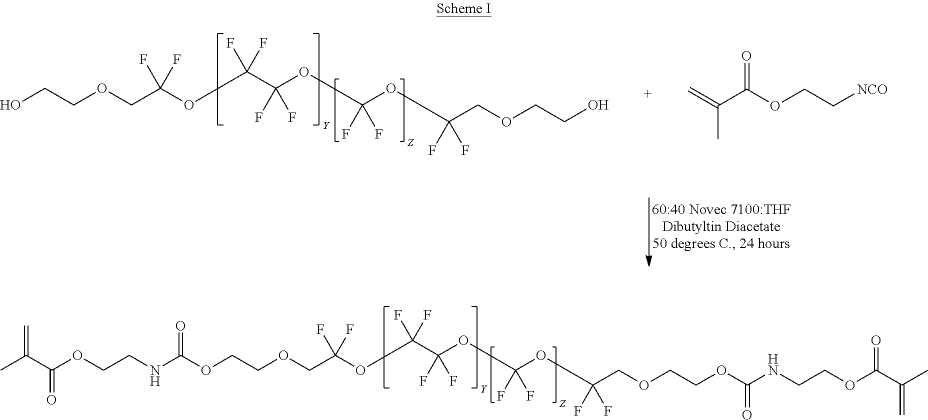

The same method can be used to synthesize compounds of the formula (II) as shown in Scheme II, below, wherein Novec™ 7100 and THF comprises a non-limiting solvent system that may be utilized to synthesize the compounds of the formula (II); and the variables X and Y are as defined above:

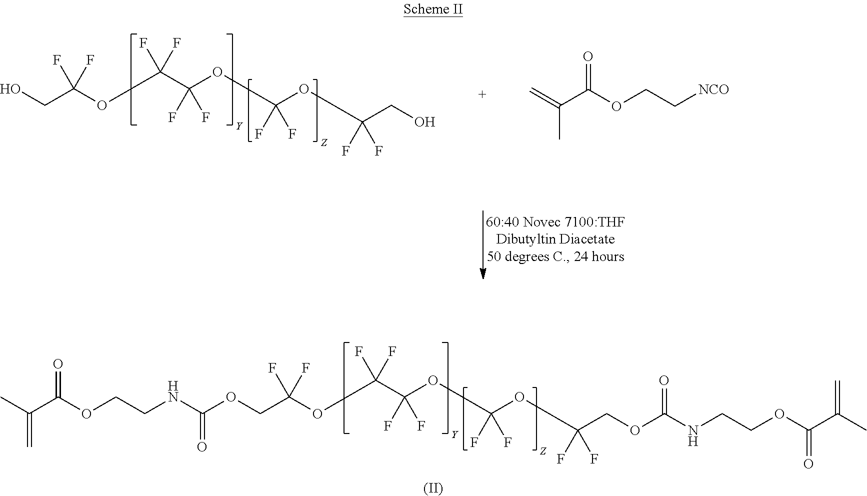

Some embodiments of the present invention also contemplate hydrophobic, cross-linkable polymers of the formula (III):

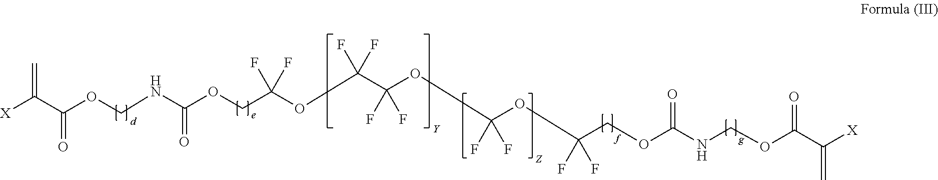

wherein Y and Z are as defined herein; X is H or C₁-C₂₀ alkyl (e.g., C₁-C₁₂, C₁-C₆, and C₁-C₄ alkyl, such as CH₃); and d, e, f, and g are each, independently, about 0 to about 5, e.g., from about 0 to about 2, from about 1 to about 4, from about 2 to about 5 or from about 3 to about 4. In some embodiments, Y and Z are each, independently, from about 10 to about 50, each X is, independently, H or C₁-C₂₀ alkyl, and d, e, f, and g are each, independently, about 0 to about 5.

In one example, compounds of the formula (I)-(III), and combinations thereof, can be cross-linked (i.e., polymerized) via radical chemistry in the presence of a radical initiator (e.g., ammonium peroxodisulfate, dibenzoyl peroxide, 2,2-Dimethoxy-2-phenylacetophenone, and mixtures thereof).

In some embodiments, the microcapsules may comprise a liquid core. In some embodiments, the liquid core comprises an active agent. In other embodiments, the liquid core comprises an organic solvent (e.g., methanol, ethanol, isopropanol, dichloromethane, ethyl acetate, chloroform, hexane, mineral oil, THF, toluene, perfluorinated solvents, olive oil, sunflower oil, etc.). In some embodiments, the organic solvent may be other than an ethyl acetate and/or perfluorinated solvents.

In certain embodiments, the liquid core comprises an emulsion. The emulsion may be preformed, or the emulsion may be not preformed. Emulsions can be any suitable emulsion including, but not limited to, water in oil or oil in water emulsions. In some embodiments, as oil phase, an organic solvent (e.g., methanol, ethanol, ethyl acetate, isopropanol, dichloromethane, chloroform, hexane, mineral oil, THF, toluene, olive oil, sunflower oil, perfluorinated solvents, etc.) can be applied with the exception of THF, methanol, isopropanol, and ethanol. In certain cases, however, the organic solvent may be or include THF, methanol, isopropanol, and ethanol. In some embodiments, the organic solvent may be an organic solvent other than ethyl acetate and/or perfluorinated solvents. In some embodiments, the emulsions can contain surfactant in the inner or outer phase, but surfactants may not be necessary.

The preformed emulsion can be formed, in some embodiments, by shaking, vortex emulsification, ultrasound emulsification, spontaneous emulsification, membrane emulsification, vibrating nozzle emulsification, high pressure homogenization, mechanical homogenization, rotor stator homogenization, magnetic stirring, mechanical stirring, static mixing, or using a microfluidic device.

In some cases, the emulsion may comprise monodisperse or heterodisperse droplets. In some embodiments, for example, the droplets may be monodisperse within an emulsion, or the droplets may have an overall average diameter and a distribution of diameters such that no more than about 5%, no more than about 2%, or no more than about 1% of the droplets have a diameter less than about 90% (or less than about 95%, or less than about 99%) and/or greater than about 110% (or greater than about 105%, or greater than about 101%) of the overall average diameter of the plurality of droplets. However, in other embodiments, the droplets may be heterodisperse or otherwise fall outside these ranges.

In some cases, there may be a relatively large number of droplets contained within a microcapsule. For example, there may be at least 5, at least 10, at least 20, at least 30, at least 50, at least 75, at least 100, at least 200, at least 300, at least 500, at least 1,000, at least 2,000, at least 3,000, at least 5,000, or at least 10,000 droplets contained within a microcapsule. The microcapsules may all have substantially the same number of droplets therein (e.g., no more than about 5%, no more than about 2%, or no more than about 1% of the microcapsules may have less than about 90%, less than about 95%, or less than about 99% and/or greater than about 110%, greater than about 105%, or greater than about 101% of the overall average number of droplets within the microcapsules), or in some cases, the microcapsules may have a range of droplet number distributions that fall outside these ranges.

In some embodiments, the ratio between viscous aqueous phase and organic solvent in the preformed emulsion can vary dependent on the application. Typical volume ratios of dispersed aqueous phase to organic solvent are: 1:0.3, 1:0.4, 1:0.5, 1:0.6, 1:0.7, 1:0.8, 1:0.9, 1:1, 1:2, 1:3, 1:4, 2:3, 2:4, 3:4, etc. However, it should be understood that the invention is not limited to only these volume ratios.

According to certain aspects, the systems and methods described herein can be used in a plurality of applications. For example, fields in which the particles and multiple emulsions described herein may be useful include, but are not limited to, food, beverage, health and beauty aids, paints and coatings, chemical separations, agricultural applications, and drugs and drug delivery. For instance, a precise quantity of a fluid, drug, pharmaceutical, or other species can be contained in a droplet or particle designed to release its contents under particular conditions. In some instances, cells can be contained within a droplet or particle, and the cells can be stored and/or delivered, e.g., to a target medium, for example, within a subject. Other species that can be contained within a droplet or particle and delivered to a target medium include, for example, biochemical species such as nucleic acids such as siRNA, RNAi and DNA, proteins, peptides, or enzymes. Additional species that can be contained within a droplet or particle include, but are not limited to, colloidal particles, magnetic particles, nanoparticles, quantum dots, fragrances, proteins, indicators, dyes, fluorescent species, chemicals, or the like. The target medium may be any suitable medium, for example, water, saline, an aqueous medium, a hydrophobic medium, or the like.

In still other embodiments, the liquid core comprises an aggressive material that would otherwise undermine the integrity of a shell made from traditional materials, such as organic solvents, acids, bases (or solutions of low or high pH), oxidizing agents, and reducing agents. In still other embodiments, the liquid core comprises at least one active agent dissolved in an organic solvent. The active agent may be at least one of a cosmetic, diagnostic agent, a pharmaceutical, an agrochemical, and a food additive.

Examples of diagnostic agents include, but are not limited to: vascular imaging agents such as those used in angiography, percutaneous coronary intervention, venography, intravenous urography (IVU), contrast-enhanced computed tomography (CT), contrast-enhanced MRI, dynamic contrast-enhanced MRI and contrast-enhanced ultrasound (CEUS), and CT or MR angiography studies; luminal agents such as those used in voiding cystourethrography (VCUG), hysterosalpinogram (HSG), barium enema, double contrast barium enema (DCBE), barium swallow, barium meal, double contrast barium meal, barium follow through, and virtual colonoscopy.

Contrast agents include, but are not limited to, imaging and/or therapeutic agents such as radiocontrast agents, thorium-based contrast agents, thorotrast, iodinated contrast agents, iodine, diatrizoate, metrizoate, ioxaglate, iopamidol, iohexyl, ioxilan, iopromide, iodixanol, barium based contrast agents, barium, barium sulfate, gadolinium-containing contrast agents, gadodiamide, gadobenic acid, gadopentetic acid, gadoteridol, gadofosveset, gadoversetamide, gadoxetic acid, gadobutrol, gadocoletic acid, gadodenterate, gadomelitol, gadopenamide, gadoteric acid, iron-oxide contrast agents, cliavist, combidex, endorem (feridex), resovist, sinerem, perflubron, optison, levovist, microbubble contrast agents, microbubbles containing fluorinated gases such as perfluorohexane and Sulfur hexafluoride, and Mangafodipir trisodium (Mn-DPDP). Examples of pharmaceuticals include, but are not limited to antibiotics, antitussives, antihistamines, decongestants, alkaloids, mineral supplements, laxatives, antacids, anti-cholesterolemics, antiarrhythmics, antipyretics, analgesics, appetite suppressants, expectorants, anti-anxiety agents, anti-ulcer agents, anti-inflammatory substances, coronary dilators, cerebral dilators, peripheral vasodilators, anti-infectives, psychotropics, antimanics, stimulants, gastrointestinal agents, sedatives, anti-diarrheal preparations, anti-anginal drugs, vasodialators, anti-hypertensive drugs, vasoconstrictors, migraine treatments, antibiotics, tranquilizers, anti-psychotics, antitumor drugs, anticoagulants, antithrombotic drugs, hypontics, anti-emetics, anti-nausants, anti-convulsants, neuromuscular drugs, hyper- and hypoglycemic spasmodics, uterine relaxants, mineral and nutritional additives, antiobesity drugs, anabolic drugs, erythropoetic drugs, antiashmatics, cough suppressants, mucolytics, anti-uricemic drugs, mixtures thereof, and the like.

Examples of agrochemicals include, but are not limited to, chemical pesticides (such as herbicides, algicides, fungicides, bactericides, viricides, insecticides, acaricides, miticides, nematicides, and molluscicides), herbicide safeners, plant growth regulators, fertilizers and nutrients, gametocides, defoliants, desiccants, mixtures thereof and the like.

Examples of food additives include, but are not limited to, vitamins, minerals, color additives, herbal additives (e.g., echinacea or St. John's Wort), antimicrobials, preservatives, mixtures thereof, and the like.

In some cases, the microcapsules can be formed using a preformed dispersion as inner phase, the shell-forming polymer dissolved in an appropriate solvent as middle phase, and a suitable surfactant dissolved in water as outer continuous phase. The inner phase may include solid particles dispersed in an organic (e.g. perfluorohexane, dichloromethane, ethanol, or ethyl acetate) or aqueous phase; the particles can include pure active agent or comprise the active agent in a matrix; e.g. gelatin, alginate, chitosan, guar, PLGA, PLA, or polycaprolactone. Methods to fabricate such particles include coacervation, spray drying, solvent evaporation, precipitation, and extrusion. Size range of dispersed active-containing particles: 20 nm-5 micrometers. However, other sizes of particles are also possible in some embodiments.

In some cases, the organic phase can contain a surfactant, stabilizing polymers (e.g. polyethylene glycol, PVP, polyethylene glycol-b-polypropylene glycol-b-polyethylene glycol, polypropylene glycol-b-polyethylene glycol-b-polypropylene glycol), or stabilizing colloidal particles (e.g. silica particles).

Volume fraction of particles within the dispersion or emulsion can range from 0.1 to 0.74. Other volume fractions are also possible.

In some embodiments, the shell of the microcapsules of some embodiments further comprises degradable particles; that is, particles that degrade over time from, e.g., being exposed to an aqueous environment (e.g., in vivo), a basic environment (e.g., pH greater than about 7, including a pH of about 12), an acidic environment (e.g., pH less than about 7), and proteolytic environment (e.g., in vivo). The degradable particles may comprise degradable nanoparticles. In some examples, the degradable particles comprise silica particles (e.g., silica nanoparticles) that have been derivatized with an agent that makes the particles more hydrophobic. Such agents include, bur are not limited to trialkoxy-C₆-C₁₈-silanes (e.g., octyltrimethoxysilane) or trihalo-C₆-C₁₈-silanes such as:

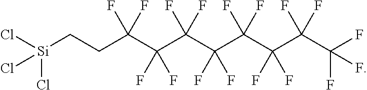 [SMILES: FC(F)(F)C(F)(F)C(F)(F)C(F)(F)C(F)(F)C(F)(F)C(F)(F)C(F)(F)CC[Si](Cl)(Cl)Cl]

Examples of other degradable particles include, but are not limited to PLA (polylacticacid), PLGA (polylactic-co-glycolic acid), inorganic particles (e.g., TiO₂), and combinations thereof.

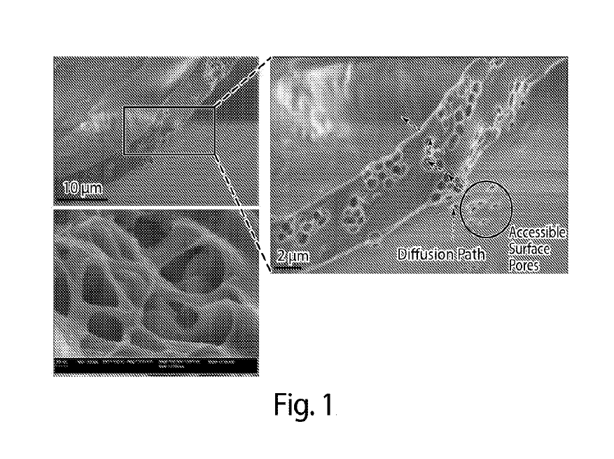

The degradable particles may degrade, over time (e.g., from about one hour to about 12 hours), thereby producing pores in the shell, wherein the pores have a dimension suitable for releasing an active present in the core of the microcapsules, by any suitable mechanism (e.g., diffusion). In some embodiments, one pore does not traverse the entire width of the microcapsule shell, but may communicate with one or more other pores, thereby forming a longer, combined pore. The molecules of active can, e.g., diffuse from the core, through one or more pore(s) in the shell, and ultimately to the space outside the shell. See FIG. 1 for example. In some embodiments, the pores have a diameter of from about 250 nm to about 900 nm, e.g., from about 300 nm to about 600 nm, from about 250 nm to about 500 nm or from about 300 nm to about 500 nm. Other pore diameters are also possible, for example, less than about 1,000 nm, less than about 500 nm, less than about 400 nm, less than about 300 nm, less than about 200 nm, less than about 100 nm, less than about 50 nm, etc. In some cases, the pore diameter may be controlled, for example, by controlling the diameter of the particles forming the pores.

In some cases, the particles are non-degradable, but can be removed from the microcapsule through various techniques, for example, through diffusion, mechanical disruption or dislodgement, or the like. In some embodiments, the particles are stable within the shell, but may be degraded by exposing the microcapsule to suitable degradation conditions. For instance, in one embodiment, the particles may be stable, but may be degraded upon exposure to suitable external conditions, such as a basic or acidic environment. In some cases, the particles are formed from a polymer that is hydrolyzable or can degrade when exposed to water or another suitable aqueous environment. For example, the particles may comprise polylactic acid, polyglycolic acid, polycaprolactone, or the like.

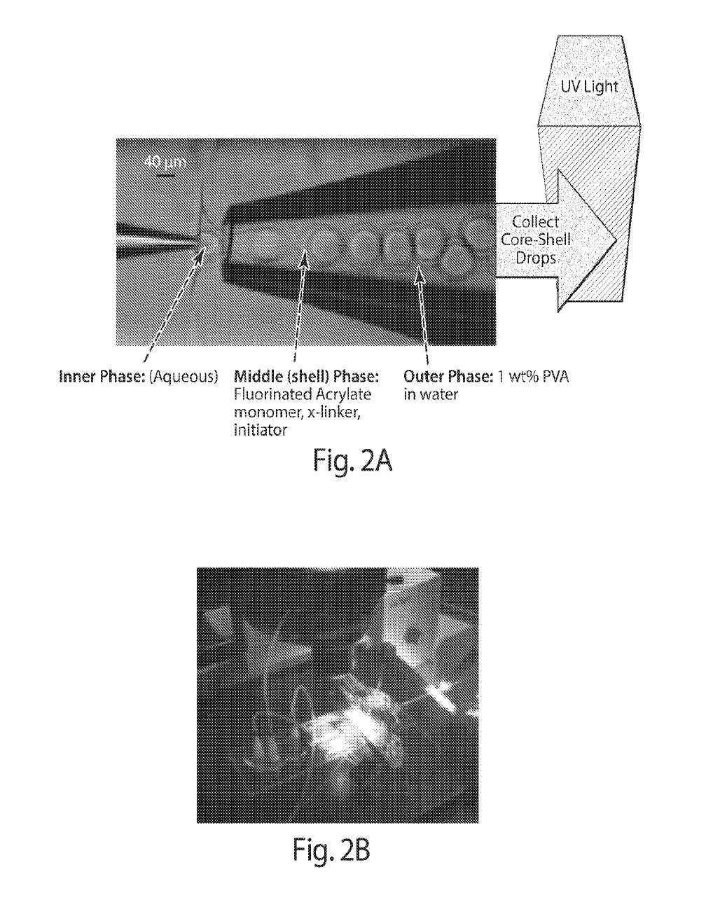

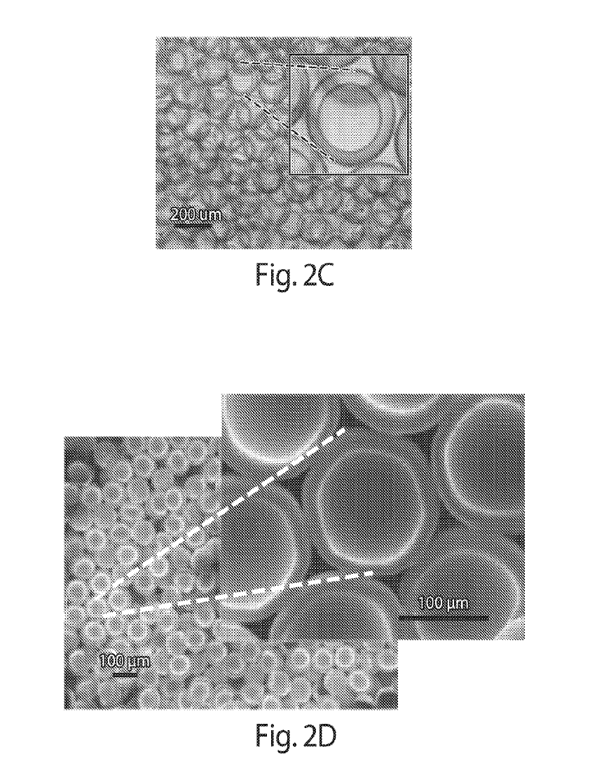

The microcapsules may be made by any suitable method. One contemplated method includes a method comprising (i) providing or obtaining a double emulsion comprising a first aqueous phase comprising a surfactant; an organic phase comprising a hydrophobic, cross-linkable (e.g., polymerizable) polymer; and a second aqueous phase optionally comprising an active; (ii) cross-linking (e.g., polymerizing) the hydrophobic, cross-linkable (e.g., polymerizable) polymer to form a hydrophobic, cross-linked (e.g., polymerized) polymeric shell substantially surrounding a core. A graphic depiction of a suitable method for making or forming the microcapsules includes the method described in FIG. 2.

Other methods of making emulsions, including double emulsions, will be known to those of ordinary skill in the art. See, for example, U.S. Pat. Nos. 9,039,273 or 7,776,927; U.S. Pat. Apl. Pub. Nos. 2014-0220350, 2013-0046030, 2012-0211084, or 2012-0199226; or Int. Pat. Apl. Pub. Nos. WO 2013/006661, WO 2012/162296, WO 2010/104604, WO 2011/028764, WO 2011/028760, WO 2008/121342, or WO 2006/096571, each incorporated herein by reference.

In some embodiments, the present invention is generally directed to forming a double emulsion where the inner fluid of the double emulsion is itself an emulsion, e.g., a pre-formed emulsion. Techniques for forming the double emulsion include any of those described herein and/or incorporated by reference. In addition, in some embodiments, the present invention is generally directed to a method of producing a double emulsion comprising an inner phase comprising a preformed emulsion, a middle phase comprising a polymer and containing the inner phase, and an outer phase containing the middle phase; and polymerizing or otherwise hardening the polymer of the middle phase to produce a microcapsule containing the emulsion.

The first aqueous phase may comprise any suitable surfactant. Examples of surfactants include, but are not limited to, polysorbates, such as “Tween 20” and “Tween 80,” and pluronics such as F68, F88, and F108; sorbitan esters; lipids, such as phospholipids including lecithin and other phosphatidylcholines, phosphatidylethanolamines, fatty acids, and fatty esters; steroids, such as cholesterol; polyvinylalcohol; and anionic surfactants, such as sodium dodecyl sulfate (SDS).

In some embodiments, the organic phase is located in between the first aqueous phase and the second aqueous phase. In some embodiments, the organic phase does not comprise an organic solvent. In other words, in some embodiments, the organic phase contains only the hydrophobic, cross-linkable polymer. In other embodiments, the organic phase contains the hydrophobic, cross-linkable polymer and whatever agent is necessary to cross-link the polymer. Such agents include catalysts (e.g., ring-opening polymerization catalysts) and initiators (e.g., free radical initiators). In some embodiments, the organic phase substantially surrounds the second aqueous phase. In some embodiments, the first aqueous phase substantially surrounds the organic phase.

The microcapsules of some embodiments of may be used in methods for delivering an active to a subject (e.g., a mammal, specifically a human) in need thereof or, in the case of agrochemicals, to an area (e.g., a field or plot) in need thereof. The methods comprise, in some embodiments, (i) providing or obtaining one or more microcapsules comprising a core and a hydrophobic, cross-linked polymeric shell, wherein the core comprises an active; and (ii) applying a trigger; wherein the trigger ruptures the one or more microcapsules, thereby delivering the active.

In embodiments where the microcapsules are delivered, the microcapsules may be delivered to the subject in need thereof or, in the case of agrochemicals, to an area in need thereof, by any suitable means. Such techniques for delivering microcapsules to a subject in need thereof include, but are not limited to, oral, peroral, parenteral, intravenous, intraperitoneal, intradermal, intramuscular, nasal, buccal, subcutaneous, rectal or topical means, for example on the skin, mucous membranes or in the eyes. In one embodiment, the technique is not subcutaneous. Techniques for delivering or depositing the microcapsules to an area in need thereof include, but are not limited to, spraying (e.g., an aqueous suspension of microcapsules) or non-spraying techniques, such as painting, flushing, deposition, or the like.

In some embodiments, the microcapsules may be combined with other pharmaceutically acceptable or agronomically acceptable excipients. Such excipients may facilitate the incorporation of microcapsules into other dosage forms (e.g., capsules, tablets, lozenges, and the like) or into, e.g., pellets for agrochemical applications.

The trigger applied to the microcapsules to rupture them may be any suitable trigger. Such triggers include, but are not limited to oxidizing stress or osmotic stress. Other suitable triggers include pH and phototriggers; reducing agents; and enzyme/enzymatic triggers. In some embodiments, applying oxidizing stress to the microcapsules includes contacting the microcapsules with or exposing the microcapsules to an oxidizing agent. Suitable oxidizing agents include, but are not limited to, silver nitrate, potassium permanganate, osmium tetroxide, peroxides, and sulfuric acid.

An osmotic stress trigger includes, but is not limited to, exposing the microcapsules to circumstances where the ionic strength outside the microcapsule is substantially less than the ionic strength inside the microcapsule (i.e., in the core). An example of such a situation includes microcapsules containing a high salt (e.g., CaCl₂) concentration (e.g., from about 1 to about 2 M salt) in the core being exposed to a significantly lower salt (e.g., about 0 to about 0.5 M) concentration outside the microcapsule.

In some embodiments, for example, the microcapsules may include a polymer that is relatively permeable to water. Thus, upon exposure to water, water is able to enter the capsules (e.g., due to the interiors of the capsules being hyperosmotic), and such water influx may ultimately trigger the microcapsules to rupture. It should also be noted that the capsules need not “shatter” or disintegrate into fragments in order to rupture; for example, a simple break, rip, hole, or tear within a wall of the microcapsule may be sufficient to allow release of actives.

In certain embodiments, the capsules may be constructed such that when exposed to a suitable trigger, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the microcapsules rupture within 30 minutes. This may be facilitated, for example, due to relatively thin shells (e.g., as discussed above), having a semipermeable shell (as discussed above), dissolution of salts, particles, or other species within the shells (e.g., weakening the shells and/or creating new transport pathways across the shell), having an interior that is off-center relative to the capsule in at least some of the capsules (e.g., such that at least a portion of the shell is thinner), or the like. Combinations of any of these and/or other approaches may also be used. In some embodiments, systems may be used to facilitate rupture of the capsules within 30 minutes, or less in some cases. For instance, rupture as discussed above may occur within 20 minutes, 15 minutes, 10 minutes, 5 minutes, 3 minutes, or 1 minute.

The following documents are incorporated herein by reference in their entirety for all purposes: U.S. Provisional Application Ser. No. 61/980,541, filed Apr. 16, 2014, entitled “Systems and methods for producing droplet emulsions with relatively thin shells”; International Patent Publication Number WO 2004/091763, filed Apr. 9, 2004, entitled “Formation and Control of Fluidic Species,” by Link et al.; International Patent Publication Number WO 2004/002627, filed Jun. 3, 2003, entitled “Method and Apparatus for Fluid Dispersion,” by Stone et al.; International Patent Publication Number WO 2006/096571, filed Mar. 3, 2006, entitled “Method and Apparatus for Forming Multiple Emulsions,” by Weitz et al.; International Patent Publication Number WO 2005/021151, filed Aug. 27, 2004, entitled “Electronic Control of Fluidic Species,” by Link et al.; International Patent Publication Number WO 2008/121342, filed Mar. 28, 2008, entitled “Emulsions and Techniques for Formation,” by Chu et al.; International Patent Publication Number WO 2010/104604, filed Mar. 12, 2010, entitled “Method for the Controlled Creation of Emulsions, Including Multiple Emulsions,” by Weitz et al.; International Patent Publication Number WO 2011/028760, filed Sep. 1, 2010, entitled “Multiple Emulsions Created Using Junctions,” by Weitz et al.; International Patent Publication Number WO 2011/028764, filed Sep. 1, 2010, entitled “Multiple Emulsions Created Using Jetting and Other Techniques,” by Weitz et al.; International Patent Publication Number WO 2009/148598, filed Jun. 4, 2009, entitled “Polymersomes, Phospholipids, and Other Species Associated with Droplets,” by Shum, et al.; International Patent Publication Number WO 2011/116154, filed Mar. 16, 2011, entitled “Melt Emulsification,” by Shum, et al.; International Patent Publication Number WO 2009/148598, filed Jun. 4, 2009, entitled “Polymersomes, Colloidosomes, Liposomes, and other Species Associated with Fluidic Droplets,” by Shum, et al.; International Patent Publication Number WO 2012/162296, filed May 22, 2012, entitled “Control of Emulsions, Including Multiple Emulsions,” by Rotem, et al.; International Patent Publication Number WO 2013/006661, filed Jul. 5, 2012, entitled “Multiple Emulsions and Techniques for the Formation of Multiple Emulsions,” by Kim, et al.; and International Patent Publication Number WO 2013/032709, filed Aug. 15, 2012, entitled “Systems and Methods for Shell Encapsulation,” by Weitz, et al.

Also incorporated herein by reference is U.S. Provisional Patent Application Ser. No. 62/063,556, filed Oct. 14, 2014, entitled “Microcapsules and Uses Thereof.”

The following examples are intended to illustrate certain embodiments of the present invention, but do not exemplify the full scope of the invention. The present invention is not limited to the examples given herein.

### Example 1

Microcapsules tailored for efficient isolation of core actives, followed by a timed release mechanism, may be made from cross-linkable perfluoropolyether (PFPE) materials. The PFPE materials, in turn, are made by synthesizing a large molecular weight monomer consisting of a PFPE block functionalized by end-cap methacrylate groups. The PFPE block confers chemical inertness and hydrophobicity to the microcapsule shell while the photo-curable acrylate groups facilitate a highly cross-linked homogeneous polymeric network. This polymeric cross-linking strategy minimizes the undesired formation shell pores, while reducing the effect of polymer swelling because of the high degree of hydrophobicity afforded by the PFPE block.

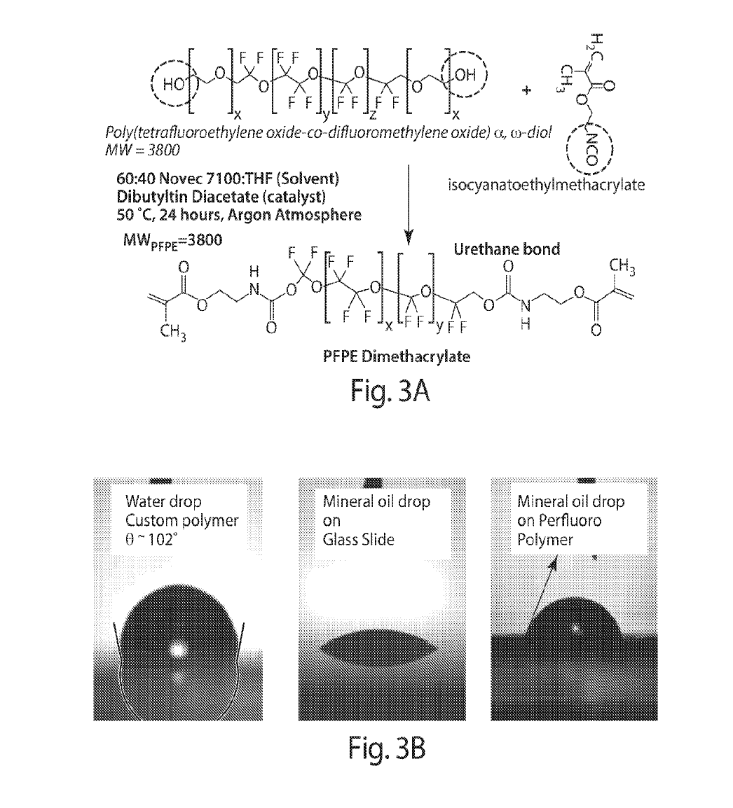

To synthesize the PFPE dimethacrylate monomer, end-capped isocyanate acrylate groups were covalently linked to a PFPE diol (number average molecular weight (Mₙ)=3,800 g/mol) using urethane chemistry in a solvent mixture as shown in FIG. 3 (panel (a)). The resulting polymer displays a contact angle with water of 102° and a large contact angle with hydrocarbon solutions, such as mineral oil, as shown in FIG. 3 (pane (b)). In some embodiments, the isocyanate acrylate capped PFPE dimethacrylate monomer displays a contact angle of from about 45° to about 105°, e.g., from about 75° to about 105°, from about 90° to about 105° or from about 100° to about 105°.

The contact angle measurements indicate that despite the presence of polar acrylic groups, the polymer is able to retain “Teflon-like” physical properties. Each monomer contains about 35 combined fluorinated ethylene and methylene groups to only 2 polar acrylate segments, thus allowing for the retention of the desirable surface properties.

To form the microcapsules, template W/O/W double emulsion drops were formed using a capillary microfluidic device as shown in FIG. 2 (panel (a)); the middle phase has the PFPE monomer encapsulating an aqueous solution and is dispersed in an aqueous surfactant continuous fluid. In situ photopolymerization was used to minimize gravitational settling effects of the density mismatched inner and middle phases to form spatially homogeneous capsule shells as shown in the photograph of FIG. 2 (panel (b)). An optical microscope image and SEM image of the resultant capsule are shown in FIG. 2 (panels (c) and (d), respectively).

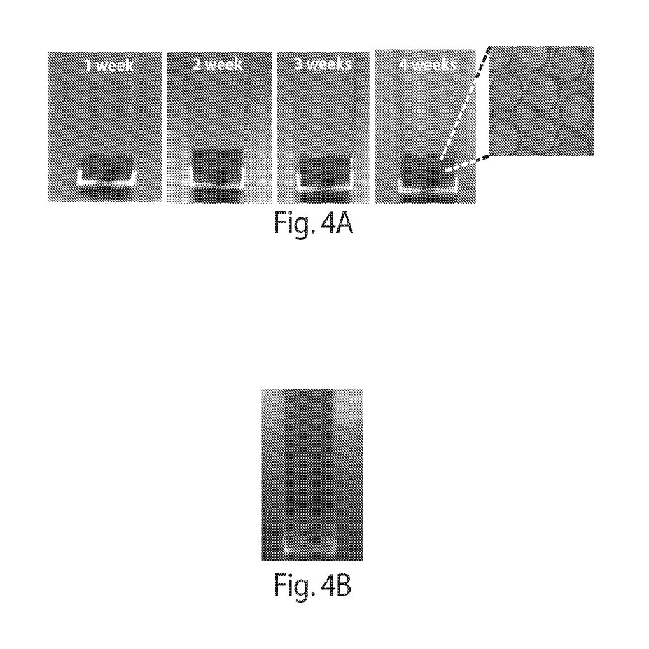

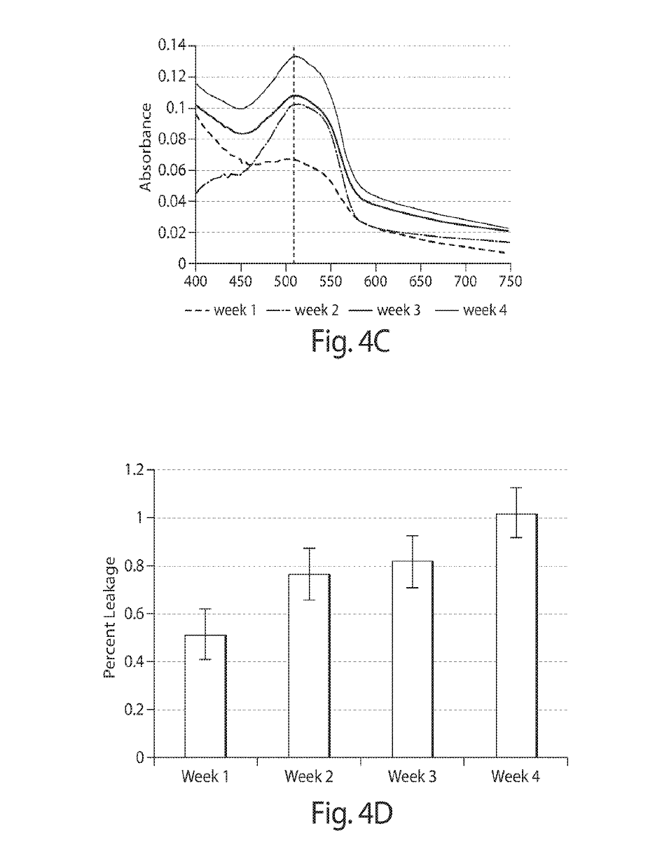

To characterize the encapsulation efficiency of the microcapsules, a 1 wt. % aqueous solution of Allura Red dye (MW=496 Da) was encapsulated in a microcapsule. UV/Vis spectroscopy was used to determine the percentage of dye leaked from the capsule core to the continuous fluid over a 4 week period. FIG. 4 (panel (a)) shows photographs of the vial containing the microcapsules taken each week. As the images indicate, the continuous fluid remains nearly transparent during the test period, indicating only a small amount of the dye has leaked from the microcapsules. At the end of the 4-week test period, the microcapsules were crushed to determine the total amount of dye encapsulated (FIG. 4, panel (b)) and the measured concentration measured each week was normalized against the total amount of dye to determine the percentage leaked. The raw UV/Vis data is shown in FIG. 4 (panel (c)). The measurements indicate that only about 1.1% of the encapsulated dye was lost during the 4 week trial as evidenced by inspection of FIG. 4 (panel (d)).

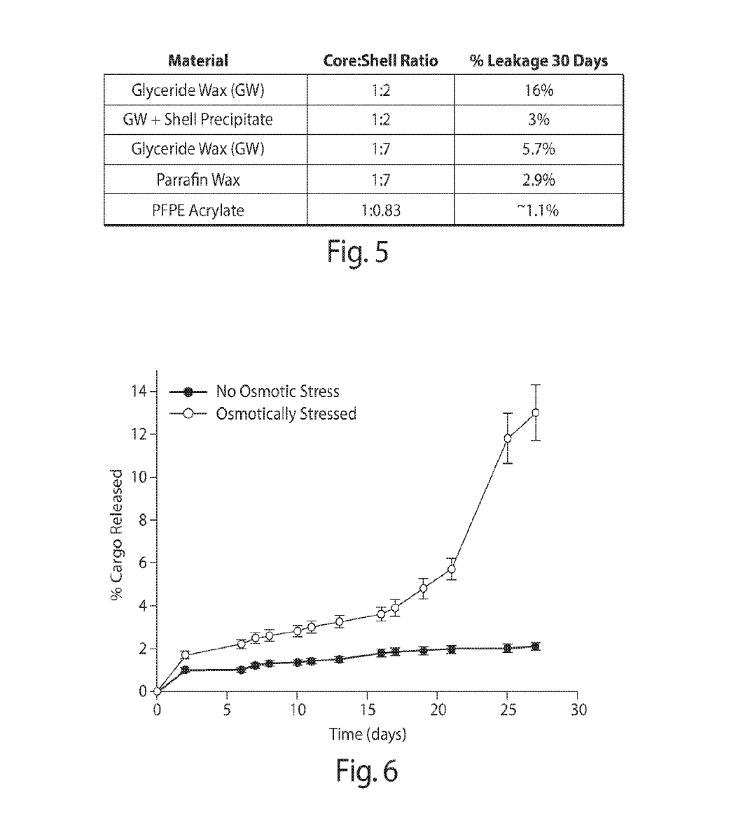

These results demonstrate a considerable improvement of encapsulation efficiency over hydrophobic wax polymeric capsules and evidenced by inspection of FIG. 5 (data from Langmuir 27: 13988-13991 (2011) and ACS Appl. Mater. Interfaces 2: 3411-3416 (2010)). These data show the percent of Allura Red dye leaked from microcapsules of the hydrophobic polymeric materials listed in FIG. 5.

The improvement in encapsulation efficiency, with increased loading capacity (2^{nd} column of the table shown in FIG. 5), may be attributed to the high degree of crosslinking afforded by the acrylate-functionalized monomer. The data from previous studies presented in FIG. 5 utilized linear wax polymers assembled by melt emulsification. Due to the random arrangement of polymer molecules during the solidification process, formation of membrane pores was generally unavoidable in such systems. That was also the case for capsule membranes formed by solvent evaporation techniques. Thus, by fabricating hydrophobic, inert capsules from cross-linkable monomers, the encapsulation efficiency was significantly improved, while maintaining the favorable physical properties afforded by hydrophobic materials.

To further demonstrate encapsulation efficiency, microcapsules were formed with an aqueous core of 1.8 M CaCl₂, and the change in conductivity of the outer fluid was measured over time to determine the amount of ions leaked from the capsules. As evidenced by inspection of FIG. 6 (filled circles) only 2.2% of the encapsulated ions leaked over a 4 week trial period. It was necessary to balance the osmotic potential of the capsules to obtain these results. This was achieved by including the appropriate osmolarity of non-conductive glucose in the continuous fluid. Osmotic stress leads to an increase in diffusion and permeability. As shown by the open circles in FIG. 6, a greater rate of leakage is observed for the microcapsules under osmotic stress.

### Example 2

To demonstrate cargo diversity, a pre-formed water-in-oil emulsion of water drops containing FITC dye dispersed in hexadecane were encapsulated in PFPE-microcapsules. Double emulsion drops were formed in which the inner phase containing the water-in-oil emulsion. In situ polymerization was used to obtain monodisperse microcapsules with a spatially homogeneous shell that contained a W/O emulsion as the core.

### Example 3

To examine the impact of organic solvents on cargo retention, PFPE microcapsules were fabricated to contain an 8 mM solution of Nile Red in toluene with a core-shell ratio of 1/0.2 v/v. These microcapsules were split into two batches. The supernatant was decanted. The microcapsules were washed with deionized water to remove the surfactant. Next, the microcapsules were suspended and incubated; the first batch in hexane and the second batch in toluene. The cumulative release of Nile Red into the supernatant was monitored over the course of 21 days using UV/Vis spectroscopy.

The results indicate a strong dependence of release kinetics on the employed exterior solution. In contrast to their behavior in water, these capsules begin a sustained release of low molecular weight hydrophobic cargo molecules immediately upon exposure to an organic continuous phase; the capsules lost 59% and 80% of the encapsulated Nile Red in hexane and toluene, respectively.

To determine the permeability coefficients of the capsules, Fick's Law was used in the case of low particle volume fraction and the data were fit to the exponential solution:

$$X ( t ) = 1 - exp ( - \frac{3 P}{a} t )$$

wherein X(t) is the fractional release of dye, a is the capsule radius, and P is the permeability coefficient. Capsules loaded with Nile Red and dispersed in toluene or hexane had permeability coefficients of 2.2 10⁻⁹ cm/s and 1.1 10⁻⁹ cm/s respectively; the increased leakage in toluene revealed the contribution of the outer phase to the observed release kinetics. However, the major contribution to the sustained leakage of encapsulated dye can be attributed to the inner carrier fluid. By ¹H-NMR measurements it was determined that toluene had a solubility of 17.6 g/100 g in the PFPE methacrylate; solubility parameters may be indicators for potential swelling by the solvent on a resultant polymer. Swelling of the shell network may lead to a lower diffusion barrier, and therefore to an accelerated leakage of encapsulated dye.

### Example 4

The toxicity of CT contrast agents can be minimized by encapsulation. Micorcapsulates having a PFPE shell and a core comprising Isovue-370 were prepared, where the microcapsules have a diameter below 3 micrometers and can be used in intravenous applications.

Isovue loaded nanocapsules by a multistep emulsification process. First, an aqueous solution of Isovue-370 (1 mL) was dispersed in PFPE-dimethacrylate (1.5 mL) that contained a radical initiator, 2,2-dimethoxy-2-phenylacetophenone (0.3 wt %) using a tip sonicator (amplitude 40%, 5 minutes). To this water-in-oil emulsion the external aqueous phase containing poly(vinyl alcohol) (10 wt %) as surfactant was added. Tip sonication (Amplitude 30%, 7 minutes) yielded a stable water-oil-water double emulsion. The middle oil phase of the double emulsion drops was solidified through photopolymerization and Isovue loaded nanocapsules were obtained.

The formulation described above yielded monomodal nanocapsules with an average diameter of 180 nm (+/−77 nm). The encapsulation efficiency was approximately 60%. The loaded nanocapsules showed improved contrast in micro-CT measurements in comparison to capsules filled with pure DI-water.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those of ordinary skill in the art, and that such modifications and variations are considered to be within the scope of this invention as defined by the appended claims.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A microcapsule comprising:
a core; and
a hydrophobic, cross-linked polymeric shell.

2. The microcapsule of claim 1, wherein the polymeric shell comprises polymers comprising cross-linked perfluoropolyether (PFPE) blocks.

3. The microcapsule of claim 2, wherein the polymeric shell comprises up to 60 mol % fluorine.

4. The microcapsule of claim 2, wherein the fluorinated polymeric shell comprises 49 mol % tetrafluoroethylene units and 49 mol % difluoromethylene units.

5. The microcapsule of claim 2, wherein the core is a liquid core.

6. The microcapsule of claim 1, wherein the core comprises an active agent.

7. The microcapsule of claim 6, wherein the active agent is at least one of a cosmetic, diagnostic agent, pharmaceutical, an agrochemical or a food additive.

8. The microcapsule of claim 1, wherein the shell further comprises degradable particles.

9. The microcapsule of claim 8, wherein the degradable particles comprise degradable nanoparticles.

10. The microcapsule of claim 9, wherein the degradable nanoparticles comprise silica nanoparticles.

11. The microcapsule of claim 1, wherein the microcapsule has a core-shell ratio of about 1:2 to about 1:0.1.

12. A method of forming a microcapsule, comprising:
(i) providing or obtaining a double emulsion comprising a first aqueous phase comprising a surfactant; an organic phase comprising a hydrophobic, cross-linkable polymer; and a second aqueous phase optionally comprising an active; and
(ii) cross-linking the hydrophobic, cross-linkable polymer to form a hydrophobic, cross-linked polymeric shell substantially surrounding a core.

13. The method of claim 12, wherein the organic phase is located in between the first aqueous phase and the second aqueous phase.

14. The method of claim 12, wherein the organic phase substantially surrounds the second aqueous phase.

15. The method of claim 14, wherein the first aqueous phase substantially surrounds the organic phase.

16. The method of claim 12, wherein the organic phase further comprises an initiator.

17. The method of claim 12, wherein the hydrophobic, cross-linkable polymer comprises cross-linkable perfluoropolyether (PFPE) blocks that are end-capped with a suitable cross-linking group.

18. The method of claim 12, wherein the hydrophobic, cross-linkable polymer comprises a compound of the formula: 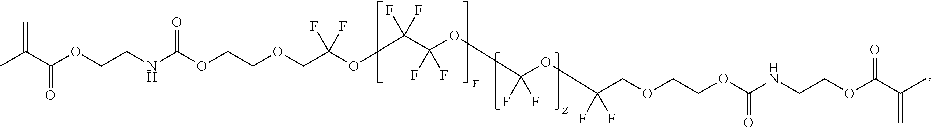
wherein Y and Z are each, independently, from about 10 to about 50; 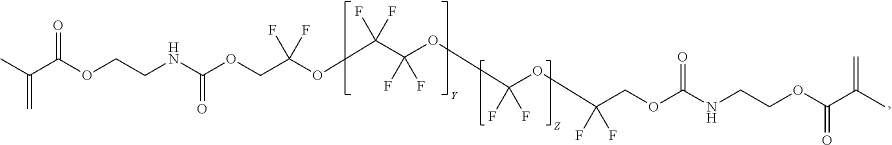
wherein Y and Z are each, independently, from about 10 to about 50; 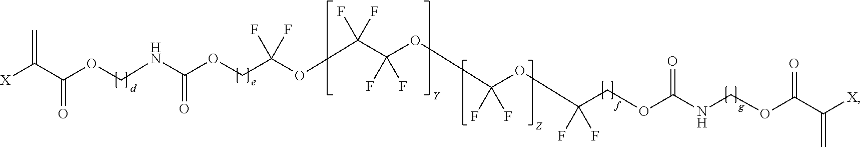
wherein Y and Z are each, independently, from about 10 to about 50, each X is, independently, H or C₁-C₂₀ alkyl, and d, e, f, and g are each, independently, about 0 to about 5; or combinations thereof.

19. The method of claim 12, wherein the cross-linked polymeric shell comprises polymers comprising cross-linked perfluoropolyether (PFPE) blocks.

20. A microcapsule comprising:
a core; and
a hydrophobic, cross-linked polymeric shell.

21. The microcapsule of claim 20, wherein the polymeric shell comprises polymers comprising cross-linked perfluoropolyether (PFPE) blocks.

22. The microcapsule of claim 21, wherein the polymeric shell comprises up to 60 mol % fluorine.

23. The microcapsule of any one of claim 21 or 22, wherein the fluorinated polymeric shell comprises 49 mol % tetrafluoroethylene units and 49 mol % difluoromethylene units.

24. The microcapsule of any one of claims 21-23, wherein the core is a liquid core.

25. The microcapsule of any one of claims 20-24, wherein the core comprises an active agent.

26. The microcapsule of claim 25, wherein the active agent is at least one of a cosmetic, diagnostic agent, pharmaceutical, an agrochemical or a food additive.

27. The microcapsule of any one of claims 20-26, wherein the shell further comprises degradable particles.

28. The microcapsule of claim 27, wherein the degradable particles comprise degradable nanoparticles.

29. The microcapsule of claim 28, wherein the degradable nanoparticles comprise silica nanoparticles.

30. The microcapsule of any one of claims 20-29, wherein the microcapsule has a core-shell ratio of about 1:2 to about 1:0.1.

31. A method of forming a microcapsule, comprising:
(i) providing or obtaining a double emulsion comprising a first aqueous phase comprising a surfactant; an organic phase comprising a hydrophobic, cross-linkable polymer; and a second aqueous phase optionally comprising an active; and
(ii) cross-linking the hydrophobic, cross-linkable polymer to form a hydrophobic, cross-linked polymeric shell substantially surrounding a core.

32. The method of claim 31, wherein the organic phase is located in between the first aqueous phase and the second aqueous phase.

33. The method of any one of claim 31 or 32, wherein the organic phase substantially surrounds the second aqueous phase.

34. The method of claim 33, wherein the first aqueous phase substantially surrounds the organic phase.

35. The method of any one of claims 31-34, wherein the organic phase further comprises an initiator.

36. The method of any one of claims 31-35, wherein the hydrophobic, cross-linkable polymer comprises cross-linkable perfluoropolyether (PFPE) blocks that are end-capped with a suitable cross-linking group.

37. The method of any one of claims 31-36, wherein the hydrophobic, cross-linkable polymer comprises a compound of the formula: 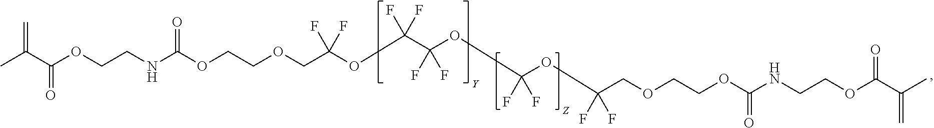
wherein Y and Z are each, independently, from about 10 to about 50; 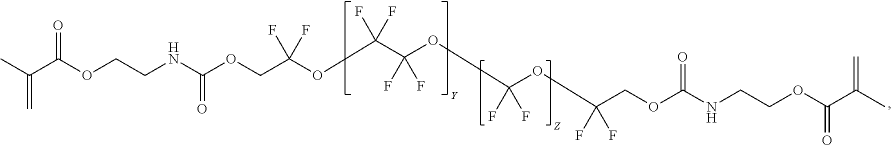
wherein Y and Z are each, independently, from about 10 to about 50; 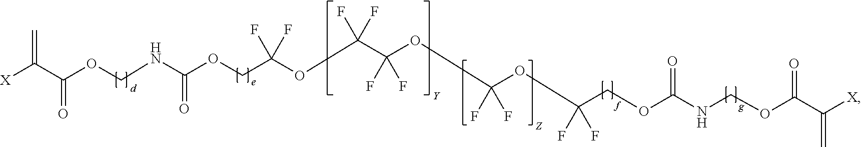
wherein Y and Z are each, independently, from about 10 to about 50, each X is, independently, H or C₁-C₂₀ alkyl, and d, e, f, and g are each, independently, about 0 to about 5; or combinations thereof.

38. The method of any one of claims 31-37, wherein the cross-linked polymeric shell comprises polymers comprising cross-linked perfluoropolyether (PFPE) blocks.

39. A microcapsule, comprising:
a core comprising an emulsion; and
a polymer shell surrounding the core.

40. The microcapsule of claim 39, wherein the emulsion is formed by shaking, vortex emulsification, ultrasound emulsification, spontaneous emulsification, membrane emulsification, vibrating nozzle emulsification, high pressure homogenization, mechanical homogenization, rotor stator homogenization, magnetic stirring, mechanical stirring, or static mixing.

41. The microcapsule of any one of claim 39 or 40, wherein the emulsion comprises an active agent.

42. The microcapsule of claim 41, wherein the active agent is at least one of a cosmetic, diagnostic agent, pharmaceutical, an agrochemical or a food additive.

43. The microcapsule of any one of claims 39-42, wherein the core is a liquid core.

44. The microcapsule of claim 43, wherein the liquid core comprises an emulsion.

45. The microcapsule of any one of claims 39-44, wherein the polymer shell comprises perfluoropolyether.

46. The microcapsule of any one of claims 39-45, wherein the polymer shell further comprises degradable particles.

47. The microcapsule of claim 46, wherein the degradable particles comprise degradable nanoparticles.

48. The microcapsule of claim 47, wherein the degradable nanoparticles comprise silica nanoparticles.

49. The microcapsule of any one of claims 39-48, wherein the microcapsule has a core-shell ratio of about 1:2 to about 1:0.1.

50. The microcapsule of any one of claims 39-49, wherein the microcapsule has a diameter of about 0.1 micrometers to about 1000 micrometers.

51. The microcapsule of any one of claims 39-50, wherein the microcapsule is substantially spherical.

52. The microcapsule of any one of claims 39-51, wherein the shell has a thickness of from about 20 nm to about 10 micrometers.

53. A method, comprising:
producing a double emulsion comprising an inner phase comprising a preformed emulsion, a middle phase comprising a polymer and containing the inner phase, and an outer phase containing the middle phase; and
polymerizing the polymer of the middle phase to produce a microcapsule containing the preformed emulsion.

54. The method of claim 53, wherein the inner phase comprises an active agent.

55. The method of claim 54, wherein the active agent is at least one of a cosmetic, diagnostic agent, pharmaceutical, an agrochemical or a food additive.

56. The method of any one of claims 53-55, wherein the polymer comprises perfluoropolyether.

57. The method of any one of claims 53-56, wherein polymerizing the polymer of the middle phase comprises cross-linking the polymer.

58. The method of any one of claims 53-57, wherein the middle phase further comprises degradable particles.

59. The method of claim 58, wherein the degradable particles comprise degradable nanoparticles.

60. The method of claim 59, wherein the degradable nanoparticles comprise silica nanoparticles.

61. The method of any one of claims 53-60, wherein the microcapsule has a core-shell ratio of about 1:2 to about 1:0.1.

62. A microcapsule, comprising:
a core; and
a polymer shell surrounding the core, the polymer shell comprising particles.

63. The microcapsule of claim 62, wherein the particles are degradable.

64. The microcapsule of any one of claim 62 or 63, wherein the degradable particles comprise degradable nanoparticles.

65. The microcapsule of claim 64, wherein the degradable nanoparticles comprise silica nanoparticles.

66. The microcapsule of any one of claims 62-65, wherein the shell comprises

67. The microcapsule of any one of claims 62-66, wherein the shell comprises up to 60 mol % fluorine.

68. The microcapsule of claim 67, wherein the shell comprises 49 mol % tetrafluoroethylene units and 49 mol % difluoromethylene units.

69. The microcapsule of any one of claims 62-68, wherein the core is a liquid core.

70. The microcapsule of claim 69, wherein the liquid core comprises an emulsion.

71. The microcapsule of any one of claims 62-70, wherein the microcapsule has a core-shell ratio of about 1:2 to about 1:0.1.

72. The microcapsule of any one of claims 62-71, wherein the microcapsule has a diameter of about 0.1 micrometers to about 1000 micrometers.

73. The microcapsule of any one of claims 62-72, wherein the microcapsule is substantially spherical.

74. The microcapsule of any one of claims 62-73, wherein the shell has a thickness of from about 20 nm to about 10 micrometers.

75. A microcapsule, comprising:
a core; and
a polymer shell surrounding the core, the polymer shell comprising cross-linked perfluoropolyether.

76. The microcapsule of claim 75, wherein the perfluoropolyether is end-capped with a cross-linking group.

77. The microcapsule of any one of claim 75 or 76, wherein the perfluoropolyether comprises a formula: 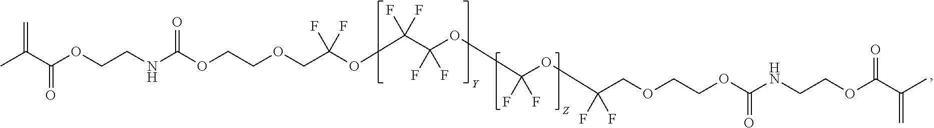
wherein Y and Z are each, independently, from about 10 to about 50; 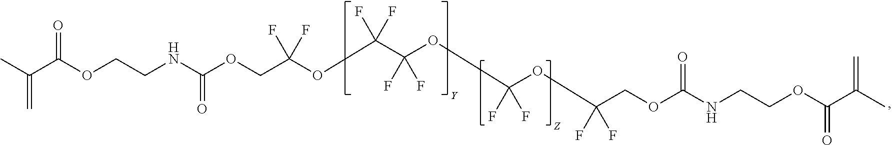
wherein Y and Z are each, independently, from about 10 to about 50; 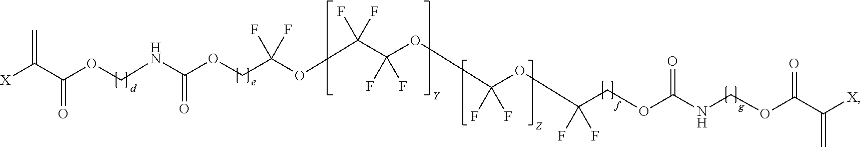
wherein Y and Z are each, independently, from about 10 to about 50, each X is, independently, H or C₁-C₂₀ alkyl, and d, e, f, and g are each, independently, about 0 to about 5; or combinations thereof.

78. The microcapsule of any one of claims 75-77, wherein the shell comprises up to 60 mol % fluorine.

79. The microcapsule of claim 78, wherein the shell comprises 49 mol % tetrafluoroethylene units and 49 mol % difluoromethylene units.

80. The microcapsule of claim 78, wherein the core is a liquid core.

81. The microcapsule of claim 80, wherein the liquid core comprises an emulsion.

82. The microcapsule of any one of claims 75-81, wherein the shell further comprises degradable particles.

83. The microcapsule of claim 82, wherein the degradable particles comprise degradable nanoparticles.

84. The microcapsule of claim 83, wherein the degradable nanoparticles comprise silica nanoparticles.

85. The microcapsule of any one of claims 75-84, wherein the microcapsule has a core-shell ratio of about 1:2 to about 1:0.1.

86. The microcapsule of any one of claims 75-85, wherein the microcapsule has a diameter of about 0.1 micrometers to about 1000 micrometers.

87. The microcapsule of any one of claims 75-86, wherein the microcapsule is substantially spherical.

88. The microcapsule of any one of claims 75-87, wherein the shell has a thickness of from about 20 nm to about 10 micrometers.
